(19) United States
(12) Patent Application Publication
(10) Pub. No.: US 2007/0244152 A1
(43) Pub. Date: Oct. 18, 2007

(54) Use of an Nk3 Antagonist for the Treatment of Bipolar Disorders

(76) Inventor: Martin T. Lowy, Research Triangle Park, NC (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/599,411

(22) PCT Filed: Mar. 25, 2005
(86) PCT No.: PCT/US05/10125

§ 371 Date: Sep. 28, 2006

Related U.S. Application Data
(60) Provisional application No. 60/556,391, filed on Mar. 25, 2004.

(51) Int. Cl. A61K 31/47

(52) U.S. Cl. 514/311

(57) ABSTRACT

This invention relates to the use of the NK3 receptor antagonist talnetant [(S)—(−)—N-(α-ethylbenzyl)-3-hydroxy-2-phenylquinoline-4-carboxamide] for treating bipolar disorder.

This invention relates to the use of NK3 receptor antagonists for treating bipolar disorder, particularly the NK3 receptor antagonist talnetant [(S)—(−)—N-(α-ethylbenzyl)-3-hydroxy-2-phenylquinoline-4-carboxamide].

Talnetant, its preparation and its use in the treatment of pulmonary disorders, disorders of the central nervous system and neurodegenerative disorders are disclosed in published International Patent application WO 95/32948. Published International Patent applications WO 97/19927, WO 97/19928, WO 99/14196 and WO 02/094187 disclose additional therapeutic utilities for talnetant, pharmaceutically acceptable salts and processes for its preparation. The above-mentioned patent applications are herein incorporated by reference as if each individual publication were specifically and individually indicated to be incorporated by reference herein as though fully set forth.

There remains the need to identify further and improved medicaments containing NK3 antagonists, particularly for the treatment or prevention of bipolar disorder.

According to a first aspect, the invention provides the use of an NK3 antagonist in the manufacture of a medicament for the treatment or prevention of bipolar disorder.

In an embodiment, the invention provides the use of talnetant, a pharmaceutically acceptable salt or solvate thereof in the manufacture of a medicament for the treatment or prevention of bipolar disorder.

Suitable pharmaceutically acceptable salts of talnetant include basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like. Preferably, talnetant is the free base. Talnetant may be crystallized or recrystallized from solvents such as aqueous and organic solvents. In such cases solvates may be formed. The invention includes within its scope stoichiometric solvates including hydrates as well as compounds containing variable amounts of water that may be produced by processes such as lyophilisation.

Hereinafter talnetant, its pharmaceutically acceptable salts and solvates defined in the first aspect of the invention are referred to simply as talnetant.

The term bipolar disorder covers all variations and sub-categories of bipolar disorder, mania, hypomania, depressed episode, rapid cycling, mixed states and manic depression, including without limitation, those categorized as shown below in the “Diagnostic and Statistical Manual of Mental Disorders” (DSM-IV-TR), Fourth Edition, edited by American Psychiatric Association:

296.00 Bipolar I Disorder, Single Manic Episode, Unspecified;

296.01 Bipolar I Disorder, Single Manic Episode, Mild;

296.02 Bipolar I Disorder, Single Manic Episode, Moderate;

296.03 Bipolar I Disorder, Single Manic Episode, Severe without Psychotic Features;

296.04 Bipolar I Disorder, Single Manic Episode, Severe with Psychotic Features;

296.05 Bipolar I Disorder, Single Manic Episode, In Partial Remission;

296.06 Bipolar I Disorder, Single Manic Episode, In Full Remission;

296.40 Bipolar I Disorder, Most Recent Episode Hypomanic;

296.40 Bipolar I Disorder, Most Recent Episode Manic, Unspecified;

296.41 Bipolar I Disorder, Most Recent Episode Manic, Mild;

296.42 Bipolar I Disorder, Most Recent Episode Manic, Moderate;

296.43 Bipolar I Disorder, Most Recent Episode Manic, Severe without Psychotic Features;

296.44 Bipolar I Disorder, Most Recent Episode Manic, Severe with Psychotic Features;

296.45 Bipolar I Disorder, Most Recent Episode Manic, In Partial Remission;

296.46 Bipolar I Disorder, Most Recent Episode Manic, In Full Remission;

296.50 Bipolar I Disorder, Most Recent Episode Depressed, Unspecified;

296.51 Bipolar I Disorder, Most Recent Episode Depressed, Mild;

296.52 Bipolar I Disorder, Most Recent Episode Depressed, Moderate;

296.53 Bipolar I Disorder, Most Recent Episode Depressed, Severe without Psychotic Features;

296.54 Bipolar I Disorder, Most Recent Episode Depressed, Severe with Psychotic Features;

296.55 Bipolar I Disorder, Most Recent Episode Depressed, In Partial Remission;

296.56 Bipolar I Disorder, Most Recent Episode Depressed, In Full Remission;

296.60 Bipolar I Disorder, Most Recent Episode Mixed, Unspecified;

296.61 Bipolar I Disorder, Most Recent Episode Mixed, Mild;

296.62 Bipolar I Disorder, Most Recent Episode Mixed, Moderate;

296.63 Bipolar I Disorder, Most Recent Episode Mixed, Severe without Psychotic Features;

296.64 Bipolar I Disorder, Most Recent Episode Mixed, Severe with Psychotic Features;

296.65 Bipolar I Disorder, Most Recent Episode Mixed, In Partial Remission;

296.66 Bipolar I Disorder, Most Recent Episode Mixed, In Full Remission;

296.80 Bipolar Disorder NOS; and

296.89 Bipolar II Disorder.

All recognized forms and variations of bipolar disorder mentioned herein are contemplated as within the scope of the present invention.

In addition to bipolar disorder, talnetant may be used for the treatment or prevention of the following diseases or conditions (as classified in the Diagnostic and Statistical Manual of Mental Disorders, 4th Edition, published by the American Psychiatric Association (DSM-IV) and/or the International Classification of Diseases, 10th Edition (ICD-10). Numbers in brackets refer to the classification code in DSM-IV):

a) Psychotic disorder including Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-induced Psychotic Disorder including the subtypes With Delusions (293.81) and With Hallucinations (293.82); and Psychotic Disorder Not Otherwise Specified (298.9);

b) Depression and mood disorders (other than bipolar disorder), including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90);

c) Anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00);

d) Substance-related disorders including Substance Use Disorders such as Substance Dependence, Substance Craving and Substance Abuse; Substance-Induced Disorders such as Substance Intoxication, Substance Withdrawal, Substance-induced Delirium, Substance-induced Persisting Dementia, Substance-Induced Persisting Amnestic Disorder, Substance-induced Psychotic Disorder, Substance-Induced Mood Disorder, Substance-Induced Anxiety Disorder, Substance-Induced Sexual Dysfunction, Substance-induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-induced Mood Disorder, Amphetamine-Induced Anxiety Disorder, Amphetamine-induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-induced Psychotic Disorder, Cocaine-Induced Mood Disorder, Cocaine-Induced Anxiety Disorder, Cocaine-induced Sexual Dysfunction, Cocaine-induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-induced Psychotic Disorder, Inhalant-induced Mood Disorder, Inhalant-induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide;

e) Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; sleep apnea and jet-lag syndrome;

f) Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50);

g) Autism Spectrum Disorders including Autistic Disorder (299.00), Asperger's Disorder (299.80), Rett's Disorder (299.80), Childhood Disintegrative Disorder (299.10) and Pervasive Disorder Not Otherwise Specified (299.80, including Atypical Autism);

h) Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-impulse Type (314.01) and Attention-Deficit/Hyperactivity Disorder Not Otherwise Specified (314.9); Hyperkinetic Disorder; Disruptive Behavior Disorders such as Conduct Disorder including the subtypes childhood-onset type (321.81), Adolescent-Onset Type (312.82) and Unspecified Onset (312.89), Oppositional Defiant Disorder (313.81) and Disruptive Behavior Disorder Not Otherwise Specified; and Tic Disorders such as Tourette's Disorder (307.23);

i) Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9);

j) Enhancement of cognition including mild cognitive impairment and the treatment of cognition impairment in other diseases such as schizophrenia, bipolar disorder, depression, other psychiatric disorders and psychotic conditions associated with cognitive impairment, e.g. Alzheimer's disease; and

k) Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

Furthermore talnetant may be used for the treatment or prevention of cognitive impairment when not associated with a psycotic disorder, for example the treatment of impairment of cognitive functions including attention, executive function, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, AIDS-related dementia or other dementia states such as Multiinfarct dementia, alcoholic dementia, hypothyroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

Talnetant may also be used as a memory and/or cognition enhancer in healthy humans with no cognitive and/or memory deficit.

Talnetant may be used as an analgesic. In particular it may be used in the treatment or prevention of traumatic pain such as postoperative pain; traumatic avulsion pain such as brachial plexus; chronic pain such as arthritic pain such as occurring in osteo-, rheumatoid or psoriatic arthritis; neuropathic pain such as post-herpetic neuralgia, trigeminal neuralgia, segmental or intercostal neuralgia, fibromyalgia, causalgia, peripheral neuropathy, diabetic neuropathy, chemotherapy-induced neuropathy, AIDS related neuropathy, occipital neuralgia, geniculate neuralgia, glossopharyngeal neuralgia, reflex sympathetic dystrophy, phantom limb pain; various forms of headache such as migraine, acute or chronic tension headache, temporomandibular pain, maxillary sinus pain, cluster headache; odontalgia; cancer pain; pain of visceral origin; gastrointestinal pain; nerve entrapment pain; sport's injury pain; dysmennorrhoea; menstrual pain; meningitis; arachnoiditis; musculoskeletal pain; low back pain e.g. spinal stenosis; prolapsed disc; sciatica; angina; ankylosing spondyolitis; gout; burns; scar pain; itch; and thalamic pain such as post stroke thalamic pain.

Talnetant may be used as anti-inflammatory agents. In particular it may be used in the treatment of inflammation in asthma, influenza, chronic bronchitis and rheumatoid arthritis; in the treatment of inflammatory diseases of the gastrointestinal tract such as Crohn's disease, postoperative gastric ileus (POI), ulcerative colitis, inflammatory bowel disease (IBD) and non-steroidal anti-inflammatory drug induced damage; inflammatory diseases of the skin such as herpes and eczema; inflammatory diseases of the bladder such as cystitis and urge incontinence; and eye and dental inflammation.

Talnetant may be used for the treatment of allergic disorders, in particular allergic disorders of the skin such as urticaria, and allergic disorders of the airways such as rhinitis.

Talnetant may be used for the treatment of emesis, i.e. nausea, retching and vomiting. Emesis includes acute emesis, delayed emesis and anticipatory emesis. NK3 antagonists, including talnetant, may be useful in the treatment of emesis however induced. For example, emesis may be induced by drugs such as cancer chemotherapeutic agents such as alkylating agents, e.g. cyclophosphamide, carmustine, lomustine and chlorambucil; cytotoxic antibiotics, e.g. dactinomycin, doxorubicin, mitomycin-C and bleomycin; anti-metabolites, e.g. cytarabine, methotrexate and 5-fluorouracil; vinca alkaloids, e.g. etoposide, vinblastine and vincristine; and others such as cisplatin, dacarbazine, procarbazine and hydroxyurea; and combinations thereof; radiation sickness; radiation therapy, e.g. irradiation of the thorax or abdomen, such as in the treatment of cancer; poisons; toxins such as toxins caused by metabolic disorders or by infection, e.g. gastritis, or released during bacterial or viral gastrointestinal infection; pregnancy; vestibular disorders, such as motion sickness, vertigo, dizziness and Meniere's disease; post-operative sickness; gastrointestinal obstruction; reduced gastrointestinal motility; visceral pain, e.g. myocardial infarction or peritonitis; migraine; increased intercranial pressure; decreased intercranial pressure (e.g. altitude sickness); opioid analgesics, such as morphine; and gastro-oesophageal reflux disease, acid indigestion, over-indulgence of food or drink, acid stomach, sour stomach, waterbrash/regurgitation, heartburn, such as episodic heartburn, nocturnal heartburn, and meal-induced heartburn and dyspepsia.

Talnetant may be used for the treatment of gastrointestinal disorders such as irritable bowel syndrome (IBS); skin disorders such as psoriasis, pruritis and sunburn; vasospastic diseases such as angina, vascular headache and Reynaud's disease; cerebral ischeamia such as cerebral vasospasm following subarachnoid haemorrhage; fibrosing and collagen diseases such as scleroderma and eosinophilic fascioliasis; disorders related to immune enhancement or suppression such as systemic lupus erythematosus and rheumatic diseases such as fibrositis; and cough.

Talnetant may be used for the treatment of neurotoxic injury which follows cerebral stroke, thromboembolic stroke, hemorrhagic stroke, cerebral ischemia, cerebral vasospam, hypoglycemia, hypoxia, anoxia or perinatal asphyxia cardiac arrest.

For the treatment of bipolar disorder, talnetant may be administered as monotherapy or in combination. When used in combination, it may be combined with one or more of the following agents to treat or prevent bipolar disease:

i) mood stabilizers such as include lithium, sodium valproate/valproic acid/divalproex, carbamazepine, lamotrigine, gabapentin, topiramate and tiagabine;

ii) antipsychotics such as typical antipsychotics (for example chlorpromazine, thioridazine, mesoridazine, fluphenazine, perphenazine, prochlorperazine, trifluoperazine, thiothixine, haloperidol, molindone and loxapine); and atypical antipsychotics (for example clozapine, olanzapine, risperidone, quetiapine, aripirazole, ziprasidone and amisulpride); and

iii) antidepressants such as serotonin reuptake inhibitors (such as citalopram, escitalopram, fluoxetine, paroxetine and sertraline); dual serotonin/noradrenaline reuptake inhibitors (such as venlafaxine, duloxetine and milnacipran); Noradrenaline reuptake inhibitors (such as reboxetine); tricyclic antidepressants (such as amitriptyline, clomipramine, imipramine, maprotiline, nortriptyline and trimipramine); monoamine oxidase inhibitors (such as isocarboxazide, moclobemide, phenelzine and tranylcypromine); and others (such as bupropion, mianserin, mirtazapine, nefazodone and trazodone).

Particular advantages associated with combinations include equivalent or improved efficacy and/or improved tolerability at doses of administration which are lower than those commonly used for the individual components where they are known for the treatment of bipolar disorder. The combinations may also provide advantages in treatment of patients who fail to respond adequately or who are resistant to treatment with certain mood stabilizing or antimanic agents which are known for the treatment of bipolar disorder.

The combinations are preferably administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of talnetant and at least one mood stabilizing or antimanic agent are within the scope of the current invention. In one embodiment of adjunctive therapeutic administration as described herein, a patient is typically stabilized on a therapeutic administration of one or more of the of the components for a period of time and then receives administration of another component.

The combinations may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

Preferably in accordance with invention, talnetant is administered orally, which will typically involve swallowing so that the compound enters the GIT. Dosage forms for oral administration include solid formulations such as tablets, capsules containing particulates or powders, sachets, vials, powders, granules, lozenges, reconstitutable powders and liquid preparations (such as suspensions, emulsions and elixirs).

Oral dosage forms of talnetant may contain further excipients such as binding agents (for example syrup, acacia, gelatin, sorbitol and tragacanth); fillers (for example lactose, sugar, maize-starch, calcium phosphate, sorbitol and glycine); tabletting lubricants (for example magnesium stearate); and disintegrants (for example starch, sodium starch glycollate and microcrystalline cellulose). In addition, the oral dosage form may contain preservatives, anti-oxidant, flavors, granulation binders, wetting agents and colorants.

Preferably the dosage form for oral administration is a tablet. Tablets may be prepared using standard technology familiar to the formulation chemist, for example by direct compression, granulation, melt congealing and extrusion. The tablet may be coated or uncoated. The tablet may be formulated to be immediate or controlled release. Controlled release formulations include delayed-, sustained-, pulsed or dual-release. Suitable tabletting excipients are described in the Handbook of Pharmaceutical Excipients, Pharmaceutical Press, 1986, published by The American Pharmaceutical Association and The Royal Pharmaceutical Society of Great Britain. Typical tabletting excipients include: carriers (for example lactose and starch), lubricating agents (for example magnesium stearate), binding agents, wetting agents, colorants, flavorings, glidants and disintegrants (for example croscarmellose sodium).

Excipients suitable for preparing liquid dosage forms include: suspending agents (for example sorbitol, syrup, methyl cellulose, gelatin, hydroxyethylcellulose, carboxymethylcellulose, aluminium stearate gel and hydrogenated edible fats); emulsifying agents (for example lecithin, sorbitan monooleate and acacia); aqueous or non-aqueous vehicles, which include edible oils (for example almond oil and fractionated coconut oil), oily esters (for example esters of glycerine and propylene glycol), ethyl alcohol, glycerine, water and normal saline; preservatives (for example methyl, propyl p-hydroxybenzoate and sorbic acid); and if desired conventional flavoring or coloring agents.

The effective dose of talnetant depends on the condition of the patient, the frequency and route of administration. A unit dose will generally contain from 20 to 1000 mg of talnetant, preferably 30 to 500 mg, most preferably 200 or 400 mg. The unit dose may be administered one or more times per day (for example 2, 3 or 4 times per day). The total daily dose for a 70 kg adult will normally be in the range 100 to 3000 mg. Alternatively the unit dose will contain from 2 to 20 mg of active ingredient and be administered in multiples, if desired, to give the preceding daily dose.

Alternatively, for acute control of symptoms, talnetant may be administered by injection (for example intravenously, intravascularly, intramuscularly, subcutaneously). The compositions may take such forms as suspensions, solutions or emulsions in oily or aqueous vehicles and may contain excipients such as suspending, stabilizing and/or dispersing agents. For administration by injection these may take the form of a unit dose presentation or as a multidose presentation preferably with an added preservative.

For long term control of symptoms, talnetant may be formulated as a depot preparation. Such long acting formulations may be administered by implantation (for example subcutaneously) or by intramuscular injection. For example talnetant may be formulated with suitable polymeric or hydrophobic materials (for example as an emulsion in an acceptable oil) or ion exchange resins or as sparingly soluble derivatives for example as a sparingly soluble salt.

All publications, including, but not limited to, patents and patent applications cited in this specification, are herein incorporated by reference as if each individual publication were specifically and individually indicated to be incorporated by reference herein as though fully set forth.

It will be appreciated that the invention includes the following further aspects. The preferred embodiments described for the first aspect extend these further aspects:
i) a method of treating or preventing bipolar disorder by administration of talnetant;
ii) talnetant for use in treating or preventing bipolar disorder.

### EXAMPLE

The following patient study may be performed to show the efficacy of talnetant in treating bipolar disorder. This study is for illustrative purposes and is not intended to limit the scope of the invention in anyway.

This example study is a multicentre, double-blind, randomized, parallel, placebo-controlled, 3-week inpatient comparison of talnetant and placebo in subjects with Bipolar I Disorder (in a recurrent manic or mixed episode). To be eligible for enrollment, a subject must meet inclusion/exclusion requirements including: 1) having a diagnosis of Bipolar I Disorder and currently experiencing a Recurrent Manic or Mixed Episode (Appendices A and B, respectively as defined in the Diagnostic and Statistical Manual of Mental Disorders, 4th edition, text revision (DSM-IV-TR) and based on the modified Structured Clinical Interview for Axis I DSM-IV Disorders (SCID) and 2) having a minimum of 20 on the YMRS. The study will last up to 42 days and will consist of 3 phases: a Screen Phase (2-7 days), a Treatment Phase (21 days), and a Follow-up Phase (14 days). After giving informed consent, completing the screening assessments, and meeting the inclusion/exclusion criteria, all subjects will enter a 2-7 day Screen Phase during hospitalization. This Phase will function: 1) as a washout period for other medications (if required) and 2) to discontinue subjects who do not continue to satisfy inclusion/exclusion criteria (e.g., based on clinical laboratory, physical examination, and/or ECG results). Following completion of the Screen Phase, subjects who continue to satisfy the inclusion/exclusion requirements will enter the 21-day Treatment Phase. The subjects will be randomized 1:1 to one of two treatment groups: 400 mg BID talnetant or placebo. The first dose of study medication will begin on the morning of Day 1 of the Treatment Phase. During the Treatment Phase, assessments will be conducted on Days 4, 7, 10, 14, 17, and 21. Subjects will remain in the hospital until the Day 7 assessments are completed. Subjects may leave the hospital anytime after completion of the Day 7 assessments if, in the Investigator's clinical judgement, they are ready for discharge and meet the community standards for level of functioning as an outpatient. Subjects who leave the hospital before Day 7 for any reason will be discontinued from the Treatment Phase, and study medication will be discontinued. The Follow-up Phase will permit safety to be assessed 14 days after the last dose of study medication. Efficacy will be assessed by using the YMRS, 21-item HAMD, CGI-S, CGI-I, and the GAF. Safety of the treatments will be evaluated by assessing vital signs, weights, clinical laboratory measures, ECGs, physical examinations, and adverse events.

1-4. (canceled)

5. A method for preventing or treating a bipolar disorder comprising administering an effective amount of a NK3 antagonist neat or admixed with a pharmaceutically acceptable carrier.

6. The method of claim 5 wherein the NK3 antagonist is talnetant.

7. The method of claim 5 wherein talnetant is in the form of the free base.

8. The method of claim 5 wherein the category of bipolar disorder one categorized in the “Diagnostic and Statistical Manual of Mental Disorders” (DSM-IV-TR), Fourth Edition, edited by American Psychiatric Association and is:
296.00 Bipolar I Disorder, Single Manic Episode, Unspecified;
296.01 Bipolar I Disorder, Single Manic Episode, Mild;
296.02 Bipolar I Disorder, Single Manic Episode, Moderate;
296.03 Bipolar I Disorder, Single Manic Episode, Severe without Psychotic Features;
296.04 Bipolar I Disorder, Single Manic Episode, Severe with Psychotic Features;
296.05 Bipolar I Disorder, Single Manic Episode, In Partial Remission;
296.06 Bipolar I Disorder, Single Manic Episode, In Full Remission;
296.40 Bipolar I Disorder, Most Recent Episode Hypomanic;
296.40 Bipolar I Disorder, Most Recent Episode Manic, Unspecified;
296.41 Bipolar I Disorder, Most Recent Episode Manic, Mild;
296.42 Bipolar I Disorder, Most Recent Episode Manic, Moderate;
296.43 Bipolar I Disorder, Most Recent Episode Manic, Severe without Psychotic Features;
296.44 Bipolar I Disorder, Most Recent Episode Manic, Severe with Psychotic Features;
296.45 Bipolar I Disorder, Most Recent Episode Manic, In Partial Remission;
296.46 Bipolar I Disorder, Most Recent Episode Manic, In Full Remission;
296.50 Bipolar I Disorder, Most Recent Episode Depressed, Unspecified;
296.51 Bipolar I Disorder, Most Recent Episode Depressed, Mild;
296.52 Bipolar I Disorder, Most Recent Episode Depressed, Moderate;
296.53 Bipolar I Disorder, Most Recent Episode Depressed, Severe without Psychotic Features;
296.54 Bipolar I Disorder, Most Recent Episode Depressed, Severe with Psychotic Features;
296.55 Bipolar I Disorder, Most Recent Episode Depressed, In Partial Remission;
296.56 Bipolar I Disorder, Most Recent Episode Depressed, In Full Remission;
296.60 Bipolar I Disorder, Most Recent Episode Mixed, Unspecified;
296.61 Bipolar I Disorder, Most Recent Episode Mixed, Mild;
296.62 Bipolar I Disorder, Most Recent Episode Mixed, Moderate;
296.63 Bipolar I Disorder, Most Recent Episode Mixed, Severe without Psychotic Features;
296.64 Bipolar I Disorder, Most Recent Episode Mixed, Severe with Psychotic Features;
296.65 Bipolar I Disorder, Most Recent Episode Mixed, In Partial Remission;
296.66 Bipolar I Disorder, Most Recent Episode Mixed, In Full Remission;
296.80 Bipolar Disorder NOS; or
296.89 Bipolar II Disorder.
